(19) United States
(12) Patent Application Publication
(10) Pub. No.: US 2006/0015180 A1
(43) Pub. Date: Jan. 19, 2006

(54) Intraocular telescope

(76) Inventors: Gholam A. Peyman, New Orleans, LA (US); Edwin Sarver, Carbondale, IL (US); John Clough, St. Petersburg Beach, FL (US); Hayden D. Beatty, Dunedin, FL (US)

Correspondence Address: Bell, Boyd & Lloyd LLC, P.O. Box 1135, Chicago, IL, 60690-1135, US

(21) Appl. No.: 11/151,978

(22) Filed: Jun. 14, 2005

Related U.S. Application Data
(63) Continuation-in-part of application No. 10/455,788, filed on Jun. 6, 2003.
(63) Continuation-in-part of application No. 10/600,371, filed on Jun. 23, 2003.
(63) Continuation-in-part of application No. 10/873,495, filed on Jun. 23, 2004.
(63) Continuation-in-part of application No. 11/038,320, filed on Jan. 17, 2005.

(51) Int. Cl. A61F 2/16

(52) U.S. Cl. 623/6.36; 623/6.34

(57) ABSTRACT

The present disclosure relates to an intraocular lens system. The lens system includes a first lens adapted to be positioned in the anterior chamber of the eye, along an optical axis, a second lens adapted to be positioned in the posterior chamber of the eye along the optical axis and in series with the first lens, and a third lens adapted to be positioned between the first and second lenses along the optical axis. The first, second and third lenses are configured to form a telescopic lens system.

### CROSS REFERENCES TO RELATED APPLICATIONS

This application is a continuation-in-part of U.S. application Ser. No. 10/455,788, filed Jun. 6, 2003, entitled “TELEDIOPTIC LENS SYSTEM AND METHOD FOR USING THE SAME,” U.S. application Ser. No. 10/600,371, filed Jun. 23, 2003, entitled “TELEDIOPTIC LENS SYSTEM AND METHOD FOR USING THE SAME”, is a continuation-in-part of U.S. application Ser. No. 10/873,495, filed Jun. 23, 2004, and entitled “BIFOCAL INTRAOCULAR TELESCOPE FOR LOW VISION CORRECTION”, and is a continuation-in-part of U.S. application Ser. No. 11/038,320, filed Jan. 17, 2005, entitled “BIFOCAL INTRAOCULAR TELESCOPE FOR LOW VISION CORRECTION”. The entire contents of each of these applications are incorporated herein by reference.

### BACKGROUND

Macular degeneration has become one of the leading causes of blindness in adults. This disease affects the central retinal area known as the macula. The macula is responsible for acute vision—i.e., vision for such things as driving or reading a newspaper. Macular degeneration can lead to a gradual or sudden loss of vision to the level of 20/200 or less. Commonly, loss of vision only affects the central macular area of about 0.25 to 4 square millimeters, and does not usually progress beyond this area, thereby leaving 95-99% of the retina unaffected. Thus, reading and driving vision can be lost, while peripheral vision remains intact. This condition is often referred to as low vision.

Most cases of macular degeneration are untreatable, although laser photocoagulation has been successful in certain instances. Telescopic systems that attach to eye glasses also have been used for many years to improve vision in patients with macular degeneration. These systems, which work by increasing the retinal image of a given object, have not been very successful because they restrict the visual field to about 11° so that normal activity is not possible. They are also large and bulky. Attempts have been made to increase the visual field by putting part of the telescope within the eye. A Galilean telescope is useful for this purpose and consists of a converging objective lens and a diverging ocular lens, which together produce a telescopic effect.

U.S. Pat. Nos. 4,666,446 and 4,581,031, both to Koziol and Peyman, and both of which are incorporated by reference herein, each disclose intraocular lenses which are implanted in the eye in place of the natural lens to redirect the rays of light to minimize the adverse affect on vision caused by the macular degeneration of the eye. For example, U.S. Pat. No. 4,666,446 discloses an intraocular lens comprising a first portion including a diverging lens and a second portion including a converging lens. The converging lens provides the eye with substantially the same focusing ability of the natural lens prior to implantation of the intraocular lens. Thus, the eye will have decreased visual acuity due to the macular degeneration, but will also have unrestricted peripheral vision. The diverging lens, on the other hand, when combined with a converging lens positioned outside of the eye (e.g., a spectacle lens), provides a magnified image with increased visual acuity but a restricted visual field. Therefore, this type of intraocular lens creates a teledioptic lens system, which provides the patient with the choice of unmagnified but peripherally unrestricted vision or magnified but peripherally restricted vision.

U.S. Pat. No. 6,197,057 to Peyman and Koziol, the entire contents of which are herein incorporated by reference, relates to a lens system that combines a high plus lens with a plus and minus intraocular lens (IOL), so that the lens system works in a manner similar to a Galilean telescope. Generally the high plus lens is outside the eye (i.e., in glasses or spectacles or in a contact lens) and the plus and minus lens is an IOL that replaces or works in conjunction with the natural lens of the patient (See FIGS. 1 and 2).

U.S. Pat. Nos. 4,074,368 and 6,596,026 B1, the entire contents of which are herein incorporated by reference, both disclose telescopic implants for implantation within an eye. These implants are designed to replace the natural lens in the eye with a telescope. They are rigid devices requiring a large incision in the eye to implant.

Although all of these systems are beneficial to patients with macular degeneration, a continuing need exists for an intraocular implant that can correct for low vision in the eye.

### SUMMARY

In one embodiment an intraocular lens system is provided. The lens system includes a first lens adapted to be positioned in the anterior chamber of the eye, along an optical axis, a second lens adapted to be positioned in the posterior chamber of the eye along the optical axis and in series with the first lens, and a third lens adapted to be positioned between the first and second lenses along the optical axis. The first, second and third lenses are configured to form a telescopic lens system.

In another embodiment, an implant is provided. The implant includes a first lens adapted to be positioned in the eye, and a second lens adapted to be positioned in the eye. The second lens has a peripheral portion with a first refractive power; and a central portion with a second refractive power. A third lens is adapted to be positioned in the eye substantially between the first and second lens, such that the first, second and third lenses form a telescopic lens system.

In another embodiment, an intraocular lens system is provided. The lens system includes a first lens adapted to be positioned in the anterior chamber of the eye, a second lens adapted to be positioned in the posterior chamber of the eye in series with the first lens, and a third lens adapted to be positioned in the eye substantially between the first and second lens. At least one strut connects the first lens and the third lens.

Additional features and advantages of the present invention are described in, and will be apparent from, the following Detailed Description of the Invention and the figures.

### BRIEF DESCRIPTION OF THE FIGURES

Referring to the drawings which form a part of this disclosure:

FIG. 1 is a cross-sectional view in side elevation of a human eye with an intraocular implant according to a first embodiment of the present invention;

FIG. 2 is an enlarged cross-sectional view in side elevation of the telescope portion of the implant shown in FIG. 1 having a plus and a minus lens therein;

FIG. 3 is a top plan view of the intraocular implant shown in FIG. 1 prior to implantation;

FIG. 4 is a side elevational view of the intraocular implant shown in FIG. 3;

FIG. 5 is an enlarged cross-sectional view in side elevation of a modified telescope portion of the present invention using diffractive lenses;

FIG. 6 is a top plan view of an intraocular implant similar to that shown in FIGS. 3 and 4, but using U-shaped haptics;

FIG. 7 is a side elevational view of the intraocular implant shown in FIG. 6;

FIG. 8 is a cross-sectional view in side elevation of a human eye with an intraocular implant according to a second embodiment of the present invention with an artificial IOL substituted for the natural lens;

FIG. 9 is a cross-sectional view in side elevation of a human eye with an intraocular implant according to a third embodiment of the present invention used with the natural lens;

FIG. 10 is a cross-sectional view in side elevation of a human eye with an intraocular implant according to a fourth embodiment of the present invention;

FIG. 11 is a cross-sectional view in side elevation of a human eye with an intraocular implant according to a fifth embodiment of the present invention;

FIG. 12 is an enlarged cross-sectional view in side elevation of the telescope portion of the intraocular implant of FIG. 11 having a plus and a minus lens therein;

FIG. 13 is an enlarged cross-sectional view in side elevation of alternative telescope portion of the present invention for use with the embodiment of FIG. 11;

FIG. 14 is an enlarged cross-sectional view in side elevation of another alternative telescope portion for use with the embodiment of FIG. 11.

FIG. 15 is a cross-sectional view in side elevation of the embodiment of FIG. 1 further including a contact lens on the cornea;

FIG. 16 is a cross-sectional view in side elevation of the embodiment of FIG. 1 further including an external spectacle;

FIG. 17 is a top plan view of a bifocal contact lens;

FIG. 18 is a perspective view of an alternative telescope portion for providing a teledioptic lens system;

FIG. 19 is an elevational side view in section of an external spectacle with an opaque portion or member blocking light from passing through the central portion of the spectacle;

FIG. 20 is an elevational side view in section of the spectacles of FIG. 19 with the opaque portion moved away from the central portion of the spectacle; and

FIG. 21 is an elevational side view of a telescopic lens system according to another embodiment of the present invention;

FIG. 22 is a front view of a telescopic lens system according to another embodiment of the present invention;

FIG. 23 is a elevational side view in section of the lens system of FIG. 22 taken along lines 23-23; and

FIG. 24 is a side view in section of an eye with the natural lens removed and the lens system of FIG. 23 implanted therein.

### DETAILED DESCRIPTION OF THE PREFFERED EMBODIMENTS

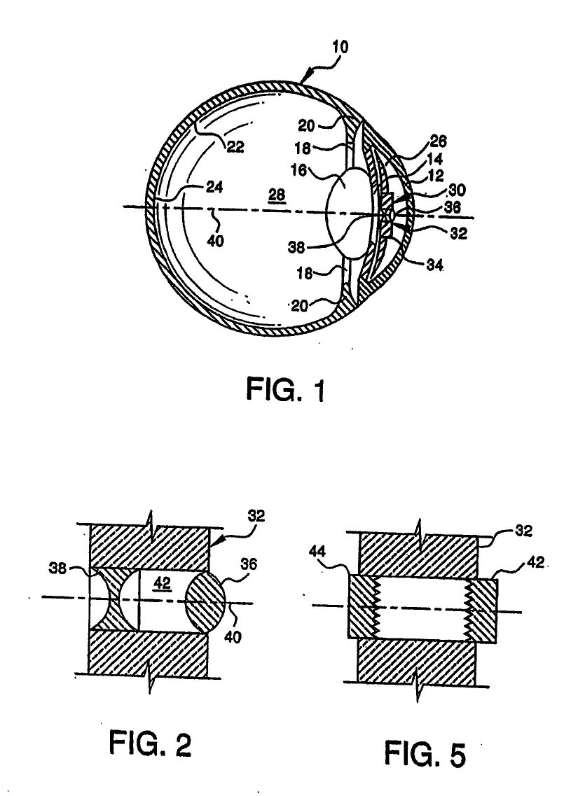

Referring to FIGS. 1-4, an eye 10 includes a cornea 12, iris 14, natural lens 16, zonular ligaments 18, ciliary sulcus 20, retina 22, and macula 24. The natural lens 16, zonular ligaments 18, and ciliary sulcus 20 divide the eye into an anterior chamber 26 and a posterior chamber 28. The macula 24 is located at the center of the retina 22, and is responsible for providing acute vision, such as that necessary for driving or reading. An intraocular telescopic lens implant 30 in accordance with the invention is implanted in the anterior chamber 26 of the eye 10. The intraocular telescopic lens implant 30 has a telescope portion 32 surrounded by a substantially transparent peripheral portion 34.

The telescope portion 32 allows light to pass therethrough and has a bi-convex converging, or plus, lens 36 and a bi-concave diverging, or minus, lens 38. The lenses 36, 38 are aligned along an optical axis 40 to form a Galilean telescope. Preferably, the lenses are about 1-2 mm in diameter. The diverging lens 38 has a refractive index between −30 and −90 diopters, as measured in water. The converging lens 36 has a refractive index between +30 and +80 diopters, as measured in water. The lenses 36, 38 are rigidly received in and fastened as necessary to the wall of a substantially cylindrical aperture 39 formed in the peripheral portion 34 of the implant 30, forming a cavity 42 therebetween. The cavity 42 is preferably vacuum sealed. The use of a vacuum in cavity 42 increases the refractive index, allowing for a smaller telescope. The lenses 36, 38 can be forced-fit or adhered to the aperture 39 so they do not move relative thereto. The lenses 36, 38 are spaced approximately 0.5 to 5 mm apart, depending on their particular optical properties, so that the telescope portion is approximately 0.3 to 5 mm thick.

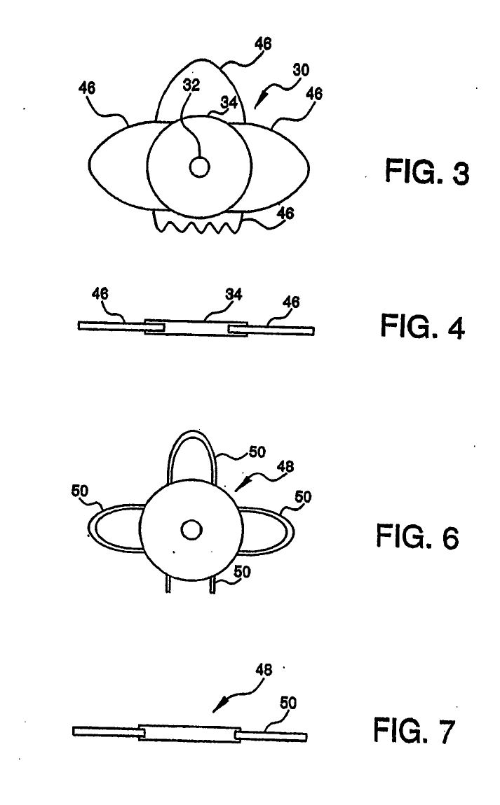

FIGS. 3 and 4 illustrate the intraocular telescopic implant 30 prior to implantation. The substantially circular peripheral portion 34 surrounding or substantially surrounding the telescope portion 32 is made of a biocompatible, transparent, optical material. Peripheral portion 34 is preferably flexible, but can be rigid or partially rigid and partially flexible or any other suitable configuration. The peripheral portion has a diameter of approximately 2 to 6.5 mm, and a thickness of approximately 0.05 to 1 mm. The peripheral portion 34 may have refractive powers to correct for refractive errors in the eye, or may have substantially no refractive powers. The peripheral portion 34 may also have varying thickness and refractive power to correct for any astigmatism in the eye. Further, the peripheral portion 34 can have multiple focal adjustments—i.e., bifocal—to correct for and provide multiple refractive corrections. Arranged around the edge of the peripheral portion 34 are from two to four haptics 46 for fastening the implant in the anterior chamber of the eye. Four haptics are shown in the illustrated embodiment, but any number of haptics may be used. With the haptics, the diameter of the implant is approximately 10-14 mm.

To implant the intraocular telescopic implant in the eye, an incision is made in the eye through the use of a microkeratome, laser, or other suitable surgical device. The implant 30 is folded or rolled up, and inserted into the anterior portion of the eye through the incision. The implant 30 is allowed to unfold or unroll, and the haptics 46 extend into the anterior chamber angle (i.e. the angle formed where the iris and the cornea meet) and fixate the implant into the anterior chamber 26 of the eye 10. Since the implant 30 is foldable, the incision is relatively small. This is beneficial because any incision to the eye can cause astigmatisms in the eye and require varying healing periods. The implant 30 may also be implanted into the posterior chamber, as shown in FIG. 10 and discussed below, or implanted into the capsular bag.

In use, the light rays that enter the eye from the central field of vision are substantially parallel to the axis 40 of the telescopic implant 30. Because they are parallel to the axis of the telescope, the rays enter the telescope and are magnified and projected onto the retina to provide enhanced acute vision for the central field of vision. At the same time, light rays from the peripheral field are unobstructed by the transparent peripheral portion 34 of the lens implant so that the patient retains unrestricted peripheral vision. Furthermore, because the peripheral portion of the implant is transparent, a doctor examining a patient's retina has an unobstructed view of the retina.

The lenses 36, 38 illustrated in FIGS. 1-2 are conventional bi-convex and bi-concave lenses. The conventional lenses are refractive lenses—i.e. they utilize refraction to modify how light propagates through the lenses to change the focal point of the lenses. The lenses in the telescopic implant 30, however, may have any desirable shape or configuration.

FIG. 5 illustrates a telescope portion 32 which uses diffractive lenses 42, 44. Diffractive lenses, such as Fresnel lenses, utilize diffraction to modify how light propagates through the lenses to change the focal point of the lenses. Diffractive lenses are advantageous because they are very thin as compared to conventional refractive lenses. Other suitable lenses include those made by ThinOptx, Inc. of Abingdon, Va. ThinOptx, Inc. manufactures intraocular lenses that are approximately 100 microns thick with +/−25 diopters of correction. Further details regarding these lenses are found in U.S. Pat. Nos. 6,666,887 and 6,096,077, which are hereby incorporated by reference in their entirety. When using technology such as this, the telescope portion can be about 2-3 mm, preferably about 2 mm thick.

The implant 30 illustrated in FIG. 1 uses haptics 46 which affix the implant into the anterior chamber angle. FIGS. 6 and 7 illustrate an implant 48 which uses alternative, substantially U-shaped haptics 50. Upon implantation, the U-shaped haptics 50 overlie the iris and can be clipped to the iris to provide added stability to the implant. One skilled in the art will recognize that although two preferred styles of haptics are specifically disclosed herein, there are a wide variety of known haptics and any suitable haptics, such as J-shaped haptics, can be used with the present invention.

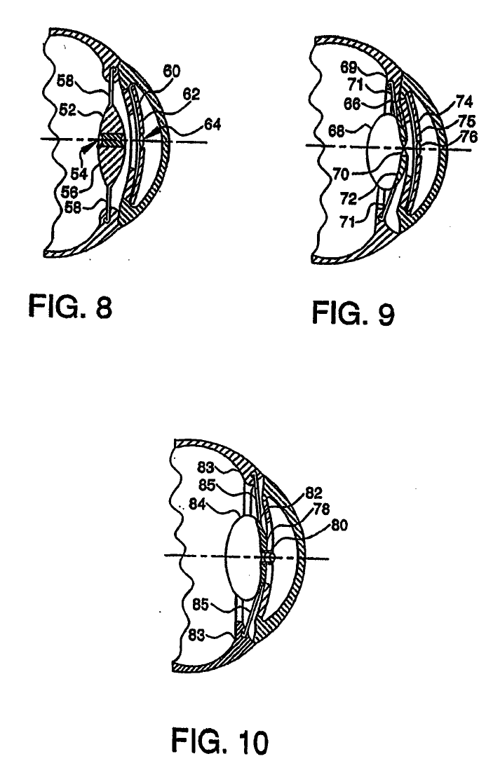

### Embodiment of FIG. 8

FIG. 8 shows a second embodiment of the present invention. In this embodiment, the natural lens of the eye is replaced with an artificial lens 52. The artificial lens 52 has a central portion 54, a peripheral portion 56, and is fastened into the posterior chamber by haptics 58. The peripheral portion 56 of the lens 52 is a generally converging lens, much like the natural lens which it replaces. The central portion 54, however, is a diverging lens with a high negative refractive index. An anterior implant 60 is located in the anterior chamber of the eye. The anterior implant 60 has a transparent peripheral portion 62 and a central portion 64. The central portion 64 is a lens with a high positive refractive index. The anterior implant central portion 64 is aligned with the artificial lens central portion 54, forming a telescope for enhancing low vision. The peripheral portion 62 has the same characteristics as peripheral portion 34 described above regarding the first embodiment of FIGS. 1-4.

### Embodiment of FIG. 9

FIG. 9 illustrates a third embodiment of present invention. In this embodiment, a first intraocular implant 66 is placed immediately adjacent the primary lens 68 and placed in the ciliary sulcus 69 of the posterior chamber by haptics 71. The illustrated primary lens 68 is a natural lens, but may also be an artificial intraocular lens. The central portion 70 of the implant 66 is a lens with a high negative refractive index and is surrounded by a peripheral portion 72, which has the same characteristics as portion 34 described above. A second intraocular implant 74 is placed in the anterior chamber of the eye. The second intraocular implant 74 has a central lens portion 76 with a positive refractive index and a peripheral portion 75 surrounding lens portion 76. Preferably, the central portions 70, 76 of the two implants 66, 74 are aligned along the main optical axis (however, these lenses can be aligned in any suitable manner), forming a telescope as discussed above regarding the embodiment of FIGS. 1-4.

### Embodiment of FIG. 10

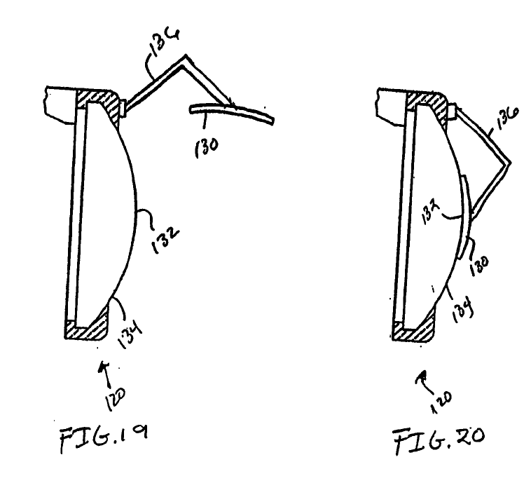

FIG. 10 shows a fourth embodiment of the present invention. In this embodiment, the intraocular implant 78 has a telescope portion 80 attached to a peripheral portion 82. The peripheral portion 82 is placed directly onto the primary lens 84 and is attached to the ciliary sulcus 83 by haptics 85. The illustrated primary lens is a natural lens, but may also be an artificial intraocular lens. The telescope portion 80 preferably is formed from a flexible material, similar to portion 34. Additionally, telescope portion can be configured as tube 80 (FIGS. 12-14) having similar characteristics as portion 34 or it can be formed as structure or telescope portion 129 having struts or extension members (FIG. 18).

As shown in FIG. 18, each strut 130, 132, 134, 136 is attached to the periphery 138 of lens 38 (in any conventional manner, such as adhesive or any other suitable means) and extends to the periphery 140 of lens 36 and attaches thereto in the same or substantially similar manner. The telescope portion 129 can have any suitable number of struts. For example, the telescope portion can have as few as one strut or as many as desirable. The struts are preferably formed from a material that can be flexible, such as the material disclosed above or portion 34 or any other suitable material. By forming the telescope portion 129 in this manner, natural fluid from the eye can flow between the lenses of the telescope portion. Additionally, the entire structure including the telescope portion 129 and peripheral portion 82 can be folded when inserted into the eye and unfolded after entry into the appropriate chamber. This flexibility allows the implant 78 to be inserted into a smaller incision in the surface of the eye, thus reducing possible damage to the eye.

When implanted, the telescope portion preferably extends through the iris; however, it is noted that the telescope portion does not necessarily need to extend through the iris and it can be situated in the eye in any suitable manner. The peripheral portion 82 has the same characteristics as portion 34 described above.

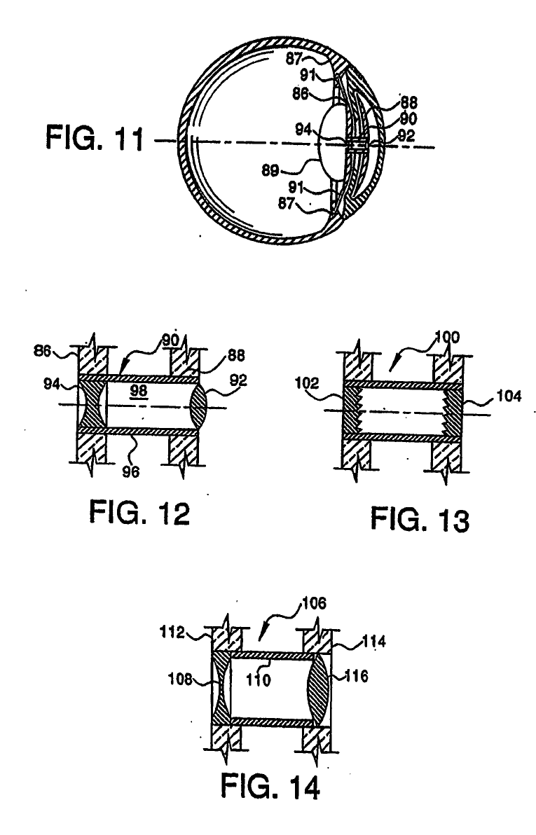

Although preferable, it is not necessary for the telescope portion 80 described in FIGS. 12-14 and telescope portion 129 described in FIG. 18 to be used with peripheral portions. For example, the telescope portion can be used with one peripheral portion, as disclosed in FIG. 10, two peripheral portions as disclosed in FIG. 11 or no peripheral portions. When used with no peripheral portions, the telescopic portion can be affixed inside the eye in any suitable manner, such as with haptics, adhesive or friction. Additionally, the telescopic portion can be affixed to the natural lens, an artificial lens or any other suitable structure (natural or artificial) inside the eye.

### Embodiment of FIGS. 11 and 12

FIGS. 11 and 12 show a fifth embodiment of the present invention. In this embodiment, a first peripheral portion 86 is located in the posterior chamber of the eye, immediately adjacent the primary lens 89. A second peripheral portion 88 is located in the anterior chamber of the eye. A telescope portion 90 is formed by a converging lens 92, a diverging lens 94, and a tubular canister 96. The tubular canister 96 is rigidly received in circular apertures in the two peripheral portions 86, 88 and connects the two peripheral portions 86, 88 through the iris. Preferably, the tubular canister and lenses 93 and 94 are flexible; however each can be rigid or any other suitable configuration.

The connection of the canister 96 at both the posterior and anterior chambers of the eye improves the stability of the telescope. The cavity 98 within tubular canister 96 may be vacuum sealed, or may contain air or water. To implant the telescope portion 90 of FIG. 12, the first peripheral portion 86 is inserted into the eye and placed in the sulcus 87 over the primary lens 89 by haptics 91. The illustrated primary lens 89 is a natural lens, but may also be an intraocular lens. The telescope portion 90 is then fastened to the first peripheral portion 86. The second peripheral portion 88 is inserted into the anterior chamber and is fastened to the telescope portion 90. The peripheral portions 86, 88 have the same characteristics as portion 34 described above. Furthermore, as described above, the telescope portion can be used with one peripheral portion, as disclosed in FIG. 10, two peripheral portions as disclosed in FIG. 11 or no peripheral portions.

FIGS. 13 and 14 show two additional telescope portions which are suitable for use in the embodiment of FIG. 11. The telescope portion 100 shown in FIG. 13 is similar to the one in FIG. 12, but uses diffractive or Fresnel lenses 102, 104 lenses instead of conventional refractive convex and concave lenses. In the telescope portion 106 shown in FIG. 14, the diverging lens 108 and canister 110 are fastened to the first peripheral portion 112 prior to implantation, and the connected pieces are implanted simultaneously. The second peripheral portion 114 and anterior lens 116 are then implanted, forming the telescope portion in situ. By assembling the telescope portion in this manner, the incision is kept to the smallest possible size.

The implantation of the lenses described herein does not necessarily need to occur during one operating procedure and can occur over a predetermined period of time (e.g., seconds, minutes, days, weeks, months or years)

Additionally, the configuration shown in FIG. 18 is suitable for this embodiment. For example, the telescope portion 129 can replace telescope portion 82. As described above, telescope portion 129 can have flexible struts that allow fluid to flow therebetween. Preferably, as described above, the struts are flexible, so that the entire lens system, including the telescope portion can be inserted into the smallest possible incision; however, the struts can be any suitable configuration (including rigid, if desired) and the telescope portion can have any number of struts desired. Any above description of telescope portion 129 is application to this embodiment.

Furthermore, the telescope portions described herein can be used with an existing IOL. For example, an existing IOLs that has high minus portions can be supplemented with an IOL (e.g., a high plus lens) that is implanted into the posterior or anterior chamber of the eye (or any other suitable portion of the eye) forming a telescopic portion, as described herein. Additionally, the supplemental IOL can be connected to the existing lens using a strut(s) or a canister as described herein. The lenses described herein are merely exemplary, and the existing and supplemental lenses can be any shape or configuration, as long as a portion of each can be combined to form a teledioptic or telescopic lens system. Examples of suitable existing IOLs are described in U.S. Pat. No. 4,666,446 to Koziol (discussed above), the entire contents of which are incorporated herein by reference.

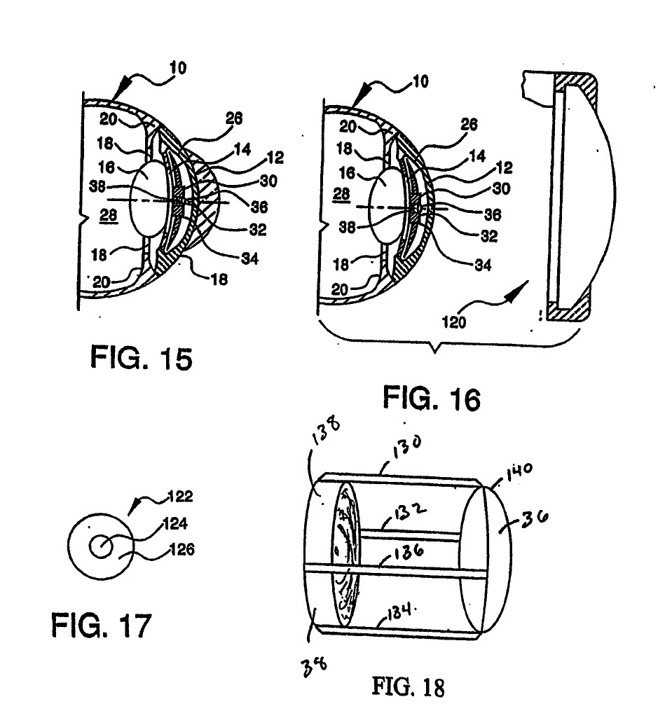

### Embodiment of FIGS. 15-17, 19 and 20

Although the invention so far has been described without the use of a supplemental lens outside the eye, it should be understood that the implants can also be used in conjunction with a supplemental lens located outside the eye. FIGS. 15 and 16 illustrate this. In FIG. 15, a supplemental plus contact lens 118 is placed on the cornea 12. In FIG. 16, a supplemental spectacle with two plus lenses 120 is placed in the visual path. In both cases, the lenses 118, 120 have a positive refractive index. The use of supplemental lenses outside the eye allows for smaller implants inside the eye. Further, the use of supplemental lenses allows the construction and operation of the implants to be tailored to particular patients' desires. For instance, many individuals have a preferable reading distance (typically between 20 and 50 cm away from the eye) and a supplemental lens allows the focal distance to be tailored to coincide with an individual's preferred reading distance. The supplemental lenses themselves can be bifocal. FIG. 17 illustrates a contact lens 122. The central 2-7 mm portion 124 of the contact lens 122 provides refractive correction for near vision.

Preferably, the peripheral portion 126 (of either the contact lens or the spectacles) provides refractive correction for far vision. The peripheral portion 126 can have any refractive properties desired. For example, the peripheral portion can be used to correct myopia, hyperopia, astigmatism, presybyopia, or any other vision error, or the peripheral portion of the lens can have no refractive properties, thus allowing a patient with acceptable peripheral vision to see with no correction (other than the telescopic central correction).

As shown in FIGS. 19 and 20, the spectacles 120 can have a removable opaque portion 130 that can be positioned over the central portion 132 of each lens. Preferably, the opaque portion 130 is substantially circular and is substantially the same size and shape as the central portion 132 of each lens.

As shown specifically, in FIG. 19, the opaque portion 130 blocks out or covers the central portion 132, thus eliminating or substantially eliminating light from passing through the central portion of the spectacle lenses and through the implanted lens(es) adapted to form a telescopic system. Substantially all light that enters the eye passes through the peripheral portion 134 of the spectacle lenses 120 and either focuses directly onto the peripheral portion of the retina or passes through the peripheral portion of an implanted lens and then onto a peripheral portion of the retina.

Opaque portion or member 130 is preferably connected to the frame of the spectacle by arm member 136. The arm member is preferably hinged to the spectacles in any suitable fashion. However, it is noted that the opaque portion can be coupled to any portion of the spectacles desired. For example, the opaque portion can be coupled to the lens, the central portion of the frame (i.e., at or near the nose portion), the peripheral portion of the frame or in any other suitable manner. Additionally, as described herein the opaque portion does not necessarily need to be coupled to the spectacles using a hinged arm and can be connected (or not) in any manner desired.

When the patient desires to focus at near objects (e.g., reading, driving, etc.) the opaque portion 130 can be flipped out of the way (FIG. 20) of the central portion 132 or removed in any other suitable manner. This allows light to pass through the central portion 132 of the spectacle lens(es) and pass through the telescopic portion of the lens system, thus enabling the patient to focus on a near object.

Additionally, if desired an opaque portion can be positioned to cover the peripheral portion 134 to eliminate substantially all light from entering the peripheral portion 134 of the spectacles 120. Spectacles 120 can have two concentric opaque portions: 1) the central opaque portion; and 2) a concentric substantially ring-shaped opaque portion that can be flipped up or down, depending on the type of vision desired by the patient. For example, if the patient desired near vision, the central opaque portion can be flipped up or moved away from the central portion of the spectacles, and the substantially ring-shaped portion could be flipped down to cover the peripheral area of the spectacle lens(es). If the patient desired to see using the peripheral portion of the spectacle lens(es) the central opaque portion could be flipped down to cover the central portion and the substantially ring-shaped portion could be flipped up or moved away from the peripheral portion of the spectacle lens(es).

It is noted that each opaque portion can be used alone or in combination with any other opaque portion, and that the opaque portions can be applied or used to cover the spectacle lens(es) in any manner desired. For example, the opaque portions can be attached to the spectacles using a lever arm 136 as shown in FIGS. 19 and 20, the opaque portion can be attached using adhesive, static, the opaque portion can be applied using any type of marking device, or the opaque portions can be any device or method that would obscure a portion or all of any type of lens, spectacle, contact or any other type.

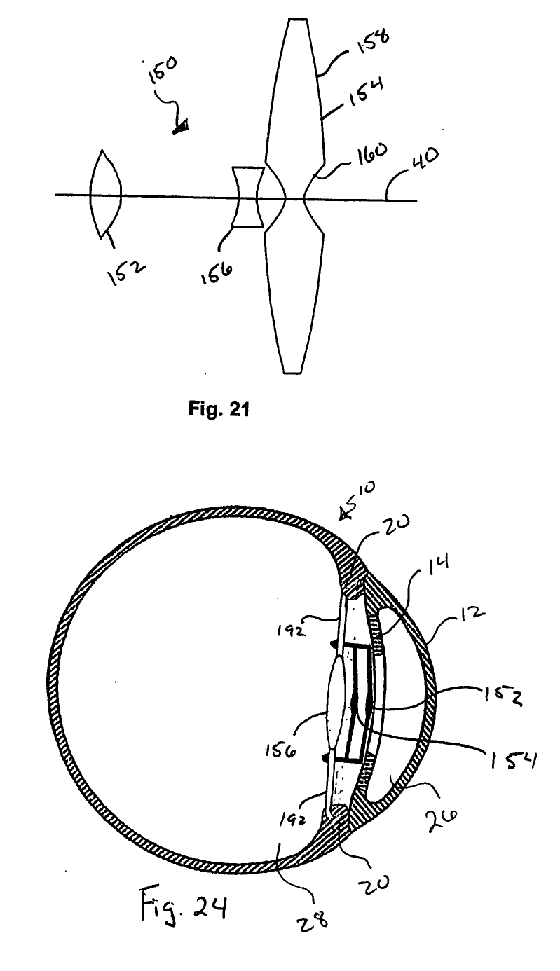

### Embodiments of FIGS. 21-24

FIGS. 21-24 illustrate additional embodiments of the present invention, wherein the telescopic intraocular lens system 150 includes at least three lenses, a first lens 152, a second lens 154 and a third lens 156. As with the above described systems, the present lens system preferable includes each of the lenses positioned substantially in series with each of the other lenses along the main optical axis of the eye.

Preferably first lens 152 is a plus lens (i.e., a biconvex asphere) and is positioned, relative the second and third lenses, closest to the cornea or the front of the eye. The first lens is preferable formed from PMMA; but can be formed from any suitable material(s). First lens 152 can also have any configuration desired and/or change or correct the refractive properties of the eye in any manner desired, that is, first lens 152 can be biconvex, biconcave, toric or any suitable combination thereof. First lens 152 preferably has a diameter between about 1.0 mm and about 1.5 mm, but can have any suitable diameter.

Second lens 154 is preferably a multifocal or bifocal lens. That is the second lens preferably has two different zones for focusing light; however, it is noted that the second lens can have any number of zones of portions capable of focusing, including one or more than two. Second lens 154 is preferably positioned, relative to the first and third lenses closest to the natural lens of the eye, if present or closest to the rear of the eye. Peripheral portion 158 of the lens 154 is a generally a converging lens (i.e., a biconvex asphere). Peripheral portion 158 preferably has a diameter about 6.0 mm; but can have any suitable diameter. The central portion 160, is a diverging lens with a high negative refractive index i.e. a biconcave lens) and has a diameter of about 1.0 mm, but can have any suitable diameter. However, it is noted that the both the central portion and the peripheral portion can be any suitable configuration desired and/or be adapted to change or correct the refractive properties of the eye in any manner desired or have no corrective properties, thus allowing light to merely pass therethrough. Second lens is preferably formed from PHMA (HEMA), but can be formed from any suitable material(s). Additionally, second lens 154 is preferably positioned in series or substantially in series with lens 152 and substantially along the main optical axis of the eye.

As shown in FIG. 21, third lens 156 is preferably a minus lens (i.e., biconcave) and is preferably positioned substantially between the first and second lenses, along the main optical axis. As with the first and second lenses, third lens can be any suitable configuration desired and/or be adapted to change or correct the refractive properties of the eye in any manner desired. Additionally, as with the first lens, third lens 156 is preferably formed from PMMA, but can be formed from any other suitable material(s). Third lens 156 preferably has a diameter between about 1.0 mm and about 1.5 mm, but can have any suitable diameter.

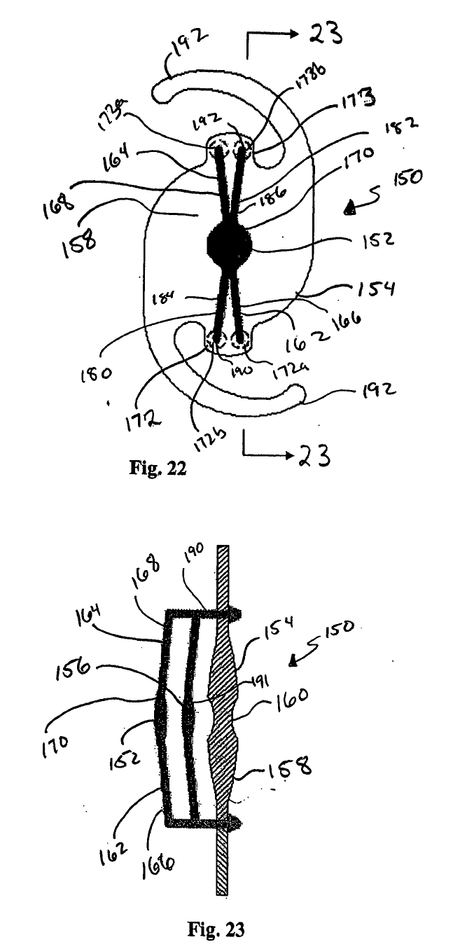

As shown in FIGS. 22-24, first lens 152 can be coupled to second lens 154 using two struts or coupling members 162 and 164. Preferably, struts 162 and 164 have a first portion 166 and 168, respectively, that each extends radially outwardly from the periphery 170 of lens 152. At about the periphery of the second lens 156 two protrusions or extensions 172 and 173 extend. The protrusions are about 180° offset from each other. Protrusion 172 has two openings 172a and 172b and protrusion 173 has two openings 173a and 173 that each extend through a respective protrusion. Second portions 174 and 176 of struts 162 and 164, respectively, extend substantially perpendicularly or at angle slightly greater than 90° to the first portion of each strut (substantially parallel to the main optical axis) and toward a respective protrusion on the second lens, coupling to the second lens at a substantially perpendicular angle. Each strut extends through a respective opening in the protrusions, allowing the struts to couple thereto. It is noted that the struts can couple the first lens to the second lens in any manner desired and do not necessarily need to be configured as described herein and/or do not need to couple to the lens as described herein.

Additionally, the first lens does not necessarily need to couple to the second lens and can couple to the third lens if desired. Furthermore, it is not necessary for the first lens to couple to the second lens using two struts and the first lens can couple to the second (and/or third) lens using as many or as few (one) struts as desired.

Third lens 156 preferably couples to second lens 154 using two struts 180 and 182. Structurally, struts 180 and 182 are substantially similar to struts 162 and 164. That is, struts 180 and 182 preferably each have a first portion 184 and 186, respectively, and a second portion 188 and 190, respectively, Each first portion extends radially outwardly from the periphery 191 of the third lens and each second portion 188 and 190 extend from a respective first portion substantially at a 90° degree angle or substantially parallel to the main optical axis and couples to the second lens through a opening or hole therein. As shown in FIG. 22, struts 180 and 182 extend from the third lens periphery slightly radially offset from struts 162 and 164. Thus, struts 180 and 182 can couple to the second lens at a different peripheral portion than struts 162 and 164.

As with struts 162 and 164 the struts can couple the third lens to the second lens in any manner desired and do not necessarily need to be configured as described herein and/or do not need to couple to the lens as described herein. Additionally, the third lens does not necessarily need to couple to the second lens and can couple to the first lens if desired. Furthermore, it is not necessary for the third lens to couple to the second lens using two struts and the third lens can couple to the second (and/or first) lens using as many or as few (one) struts as desired.

Extending from the periphery of second lens 154 are haptics 192. Although two J-shaped haptics are shown, the present device can have nay number of haptics and the haptics 192 can be any suitable configuration desired. Additionally, any or all of lenses 152, 154 and 156 can have any number of haptics extending thereof, or can be positioned and/or coupled inside of the eye in any manner desired.

As shown in FIG. 24, intraocular lens system 150 is positioned in the posterior chamber of the eye and replaces the natural lens of the eye. Preferably haptics 192 couple the lens system to the eye by piercing the ciliary sulcus 20 of the eye. However, as stated above the lens system can be positioned in the eye in any manner desired. Each of the lenses 152, 154 and 156 is preferably positioned substantially centered around the main optical axis of the eye in series with each other lens, forming a telescopic or teledioptic lens system.

This system type of system allows light traveling through the peripheral portion of the eye to be focused on the retina by the peripheral area of the of the second lens and/or the natural and/or an artificial lens and light traveling through the central portion of the cornea to be magnified by the series of lenses and/or the natural and/or an artificial lens, thus forming a bifocal or multifocal lens system. More specifically, this type of lens system allows the patient to view far objects and near objects without the aid of external lenses. However, it is noted that this type of lens system is suitable for use with external lenses (e.g., glasses or contacts), if desired.

Additionally it is noted that the lens system described herein can be used to supplement or to replace the natural lens of the eye. Additionally, the system described herein is not limited to be positioned as shown herein, that is, all lenses positioned in the posterior chamber. Each lens can be positioned in either the anterior or posterior chamber of the eye, or positioned in the pupil spanning both the anterior and the posterior chambers. For example, (1) first lens 152 can be positioned in the anterior chamber and second lens 154 and third lens 156 can be positioned in the posterior chamber; (2) the first and third lenses can be positioned in the anterior chamber and the second lens can be positioned in the posterior chamber; or (3) the first, second and third lenses can each be positioned in the anterior chamber.

In examples (1) and (2) of the above paragraph, it may be beneficial to couple the first lens directly to the third lens and/or the third lens directly to the second lens. Furthermore, the coupling member or struts in such a case can be configured such that they can pass though the pupil and not the iris, see for example, FIG. 18. However, it is noted that the lenses can couple to each other in any manner desired (including passing through the iris) and also that if desired the lenses do not need to be coupled together but can merely be positioned within the eye at the appropriate position relative to each other lens.

### EXAMPLES

The following tables show specific examples for the dimensions and design of an intraocular lens system according to the present invention. These examples were evaluated on an axis and a small field angle in 555 nm light and conditions within the eye (35° C. and surrounded by media with index of refraction of 1.336). The in situ power of the peripheral part of the primary IOL (or for example, lens 154) is 20 D. The approximate angular magnification is 3× at a distance of 50 cm compared to an equivalent eye with a 20 D IOL.

3×/20 D Intraocular Telescope - 50 cm reading distance

| Surface | Radius(mm) | Conic K | Material | Diam(mm) | Thickness(mm) |
|---|---|---|---|---|---|
| 152 Anterior | 1.5 | −1.659937 | PMMA | 1.5 | 0.6 |
| 152 Posterior | −0.75 | −1.659937 | 1.336 | 1.5 | 2.0 |
| 156 Anterior | −0.707385 | −5.180637 | PMMA | 1.0 | 0.3 |
| 156 Posterior | 0.707385 | −5.180637 | 1.336 | 1.0 | 0.5 |
| 154 cent S0 | −0.530481 | 0 | PHMA | 1.0 | 0.3 |
| 154 cent S1 | 0.530481 | 0 | 1.336 | 1.0 | |
| 154 periph S0 | 12.215 | 0 | PHMA | 6.0 | |
| 154 periph S1 | −12.215 | 0 | 1.336 | 6.0 | |

Note:
K = −e²

3×/20 D Intraocular Telescope (0.5 mm space between third lens and
second- slightly larger angular magnification) 50 cm reading distance

| Surface | Radius(mm) | Conic K | Material | Diam(mm) | Thickness(mm) |
|---|---|---|---|---|---|
| 152 Anterior | 1.5 | −1.638534 | PMMA | 1.5 | 0.6 |
| 152 Posterior | −0.75 | −1.638534 | 1.336 | 1.5 | 2.0 |
| 156 Anterior | −0.604687 | −3.024514 | PMMA | 1.0 | 0.3 |
| 156 Posterior | 0. 604687 | −3.024514 | 1.336 | 1.0 | 1.0 |
| 154 cent S0 | −0.596196 | 0 | PHMA | 1.0 | 0.3 |
| 154 cent S1 | 0. 596196 | 0 | 1.336 | 1.0 | |
| 154 periph S0 | 12.215 | 0 | PHMA | 6.0 | |
| 154 periph S1 | −12.215 | 0 | 1.336 | 6.0 | |

Note:
K = −e²

The following table illustrates an example with a 25 cm reading distance.

3×/20 D Intraocular Telescope (0.5 mm space between the third lens and the
second lens - slightly larger angular magnification) 25 cm reading distance

| Surface | Radius(mm) | Conic K | Material | Diam(mm) | Thickness(mm) |
|---|---|---|---|---|---|
| 152 Anterior | 1.5 | −1.635769 | PMMA | 1.5 | 0.6 |
| 152 Posterior | −0.75 | −1.635769 | 1.336 | 1.5 | 2.0 |
| 156 Anterior | −0.619793 | −3.112455 | PMMA | 1.0 | 0.3 |
| 156 Posterior | 0. 619793 | −3.112455 | 1.336 | 1.0 | 1.0 |
| 154 cent S0 | −0.601335 | 0 | PHMA | 1.0 | 0.3 |
| 154 cent S1 | 0. 601335 | 0 | 1.336 | 1.0 | |
| 154 periph S0 | 12.215 | 0 | PHMA | 6.0 | |
| 154 periph S1 | −12.215 | 0 | 1.336 | 6.0 | |

These examples are not meant to limit the scope of the invention and are merely to facilitate understanding of the invention. The intraocular telescope embodiments described herein can have any suitable dimensions, sizes or configurations suitable for correction and/or changing the refractive properties of the eye.

It should be understood that various changes and modifications to the presently preferred embodiments described herein will be apparent to those skilled in the art. Such changes and modifications can be made without departing from the spirit and scope of the present invention and without diminishing its intended advantages. It is therefore intended that such changes and modifications be covered by the appended claims.

The invention is claimed as follows:

1. An intraocular lens system, comprising:
a first lens adapted to be positioned in the anterior chamber of the eye, along an optical axis;
a second lens adapted to be positioned in the posterior chamber of the eye along the optical axis and in series with the first lens; and
a third lens adapted to be positioned between said first and second lenses along the optical axis;
said first, second and third lenses configured to form a telescopic lens system.

2. An intraocular lens system according to claim 1, wherein
said second lens includes
a peripheral portion with a first refractive power; and
a central portion with a second refractive power.

3. An intraocular lens system according to claim 2, wherein
said first refractive power is a plus power and said second refractive power is a minus power.

4. An intraocular lens system according to claim 3, wherein
said first lens is a plus lens and said second lens is a minus lens.

5. An intraocular lens system according to claim 1, wherein
said first lens is coupled to said third lens.

6. An intraocular lens system according to claim 5, wherein
said second lens is coupled to said third lens.

7. An intraocular lens system according to claim 1, wherein
said second lens abuts said third lens.

8. An intraocular lens system according to claim 1, wherein
said second lens is configured to be coupled to the natural lens of the eye.

9. An implant, comprising:
a first lens adapted to be positioned in the eye;
a second lens adapted to be positioned in the eye, and having
a peripheral portion with a first refractive power; and
a central portion with a second refractive power; and
a third lens adapted to be positioned in the eye substantially between the first and second lens, such that the first, second and third lenses form a telescopic lens system.

10. An implant according to claim 9, wherein
said first refractive power is a plus power and said second refractive power is a minus power.

11. An implant according to claim 10, wherein
said first lens is a plus lens and said second lens is a minus lens.

12. An implant according to claim 9, wherein
said first lens is coupled to said third lens.

13. An implant according to claim 12, wherein
said second lens is coupled to said third lens.

14. An implant according to claim 9, wherein
said second lens abuts said third lens.

15. An implant according to claim 9, wherein
said second lens is configured to be attached to the natural lens of the eye.

16. An intraocular lens system, comprising:
a first lens adapted to be positioned in the anterior chamber of the eye;
a second lens adapted to be positioned in the posterior chamber of the eye in series with the first lens;
a third lens adapted to be positioned in the eye substantially between the first and second lens; and
at least one strut connecting said first lens and said third lens.

17. An intraocular lens system according to claim 16, wherein
said second lens includes
a peripheral portion with a first refractive power; and
a central portion with a second refractive power.

18. An intraocular lens system according to claim 17, wherein
said first refractive power is a plus power and said second refractive power is a minus power.

19. An intraocular lens system according to claim 18, wherein
said first lens is a plus lens and said third lens is a minus lens.

20. An intraocular lens system according to claim 16, wherein
said second lens is coupled to said third lens.

21. An intraocular lens system according to claim 16, wherein
said third lens abuts said second lens.

22. An intraocular lens system according to claim 16, wherein
said second lens is configured to be attach to the natural lens of the eye.
